(19) United States
(12) Patent Application Publication
(10) Pub. No.: US 2024/0282419 A1
(43) Pub. Date: Aug. 22, 2024

(54) TECHNIQUE FOR SENSOR DATA BASED MEDICAL EXAMINATION REPORT GENERATION

(71) Applicant: Siemens Healthineers AG, Forchheim (DE)

(72) Inventors: Oladimeji Farri, Upper Saddle River, NJ (US); Sanjeev Kumar Karn, Plainsboro, NJ (US); Rikhiya Ghosh, Livingston, NJ (US)

(21) Appl. No.: 18/442,167

(22) Filed: Feb. 15, 2024

(30) Foreign Application Priority Data
Feb. 22, 2023 (EP) 23157914.5

(51) Int. Cl. G16H 15/00; G06F 16/34; G06F 16/36; G10L 15/26; G16H 10/60

(52) U.S. Cl. CPC G16H 15/00; G06F 16/345; G06F 16/367; G10L 15/26; G16H 10/60

(57) ABSTRACT

A technique for generating a medical examination report from sensor data of a medical examination is provided. Sensor data from a set of sensors are received in relation to the medical examination. The sensors include a microphone. The sensor data include audio data with utterances by a medical professional and patient representative. The sensor data are processed, with the audio data transformed into text. A verbatim report of the medical examination is generating. The verbatim report includes each excerpt of the text assigned to the medical professional or to the patient representative. The verbatim report is converted into a summarizing medical examination report. The summary comprises vocabulary, and/or text excerpts, by accessing a predetermined ontology database. The medical examination report is stored in an electronic medical report database.

### RELATED APPLICATION

This application claims the benefit of EP 23157914.5, filed on Feb. 22, 2023, which is hereby incorporated by reference in its entirety.

### BACKGROUND

Radiation oncology conventionally involves significant documentation of patient encounters during the courses of radiotherapy. Relevant patient data need to be extracted and populated in cancer registries to aid surveillance, clinical trial recruitment, and downstream epidemiological research. Documentation of patient encounters and populating cancer registries are conventionally manual, repetitive, and laborious tasks that present a sizeable administrative burden for radiation oncology teams. Studies have shown that physicians in general spend twice as much time documenting compared to direct patient interaction, which has resulted in physician burnout in many cases. Moreover, manual documents by the physicians are prone to be incomplete, and/or at least partially incorrect.

The introduction of technology that can listen-in on the interactions during the patient encounter (e.g., voice activated smart speakers in the visit room) has allowed for automated generation of transcripts reflecting these encounters. However, the conventional verbatim transcription of the entire dialogue by such speech recognition technology does not yield comprehensible clinical notes, as verbatim transcriptions are conventionally long, verbose, topic-wise entangled and often uninterpretable. Contrarily, real-world visit notes are terse and written in such a way that physicians can comprehend the content. Therefore, there is a need to contextually and constructively develop the clinical documents out of voice-automated clinical transcripts in a way that makes the contents actionable for the physician.

Furthermore, the privacy concerns associated with listening-in on physician-patient interactions cannot be understated, especially around institutional privacy concerns, i.e., about the way personal data and/or patient data is accessed and persisted by the company providing the smart speaker, third parties that may need to use and/or process the data, and the government's oversite around the data. Given the recent push for “privacy by design” involving visual representations of how, and which, personal data and/or patient data will be processed, there is a need to follow such guidelines and ensure transparency.

Meeting summarization, a natural language processing (NLP) task, requires generating a concise and easily consumable summary out of key utterances from audio recordings, also transcripts and video, of a multi-participant meeting. Approaches for meeting summarization broadly fall under two categories: extractive (based on selection of relevant original and/or verbatim utterances) and abstractive (further compressing and/or paraphrasing the relevant original utterances for better contextual output).

Several conventional solutions have been implemented to minimize the documentation and clerical burden experienced by physicians, and by extension radiation oncologists. These include the use of medical scribes and transcription services. Moreover, the more recent use of voice recognition via dictation software for documentation in the electronic health record (EHR) has not been shown to improve the time and effort invested by physician in clerical and administrative tasks. However, issues with system integration and workflow adaptation have emerged as barriers to using automated speech recognition for EHR documentation while avoiding patient safety-related errors.

With respect to the use of meeting (or dialogue) summarization techniques in medicine, a pipeline for automated report generation from the interactions between general practitioners (GPS) and patients has been developed. The pipeline relies on information extraction (in the form of triples) with subsequent mapping to ontologies or clinical guidelines, and report generation is based on the normalized triples. However, the quality of the reports is significantly affected by several factors including the triple extraction errors and incompleteness of the ontology.

### SUMMARY AND DESCRIPTION

It is therefore an object to provide a solution for, in particular automatically and/or in a time-saving manner, generating an accurate and/or complete medical examination report of a medical examination. Alternatively, or in addition, it is an object to ensure data privacy of patient data when, e.g., automatically, generating an accurate and/or complete medical examination report.

This object is solved by a method for generating a medical examination report from sensor data of a medical examination, by a computing device (computer), by a (e.g., distributed) system, by a computer program (also denoted as computer program product), and a non-transitory computer-readable medium. Advantageous aspects, features and embodiments are described in the claims and in the following description together with advantages.

In the following, the solution is described with respect to the claimed method as well as with respect to the claimed computing device. Features, advantages, and/or alternative embodiments herein can be assigned to the other claimed objects (e.g., the system, the computer program or a non-transitory computer-readable medium), and vice versa. In other words, claims for the computing device, and/or for the (e.g., distributed) system, can be improved with features described or claimed in the context of the method, and vice versa. In this case, the functional features of the method are embodied by structural units of the computing device, and/or of the (e.g., distributed) system, and vice versa, respectively.

As to a method aspect, a (in particular computer implemented) method for generating a medical examination report from sensor data of a medical examination is provided. The method includes an act of receiving sensor data from a set of sensors in relation to a medical examination. The set of sensors include at least one microphone. The sensor data include at least audio data (e.g., captured by the at least one microphone). The audio data include utterances by a medical professional and a patient representative participating in the medical examination.

The method further includes an act of processing the received sensor data according to the type of sensors. The processing includes at least transforming the audio data into text (e.g., initially without distinguishing between different speakers, in particular without distinguishing if an utterance stems from the medical professional or the patient representative). The method further includes an act of generating a verbatim report of the medical examination based on the processed sensor data. The verbatim report includes each excerpt of the text being assigned to the medical professional or the patient representative. The method further includes an act of converting the generated verbatim report into a medical examination report. The medical examination report includes an (e.g., abbreviated) summary of the verbatim report. The summary includes vocabulary, and/or text excerpts, by accessing a predetermined ontology database in relation to a medical field of the medical examination (e.g., a radiological oncology database).

The method still further includes an act of storing the medical examination report in an electronic medical report database.

Providing a medical examination report (also denoted as meeting summarization) may be explored under the unimodal (e.g., only using audio data, also denoted as verbal information) and/and multi-modal (e.g., including verbal and non-verbal, such as video and/or motion, information) settings. Due to the radiology oncology visit being an example of a medical examination (also denoted as meeting) between multiple participants and/or actors (e.g., patient, radiation oncologist, and/or other individuals present in the visit room, e.g., patient's relatives; briefly summarized also as patient representative and medical professional), it can be advantageous to automatically generate medical examination report (also: meeting summarization, and/or visit note), which are meaningful, accurate (also: correct), and/or complete.

By the generating and the storing of the, in particular accurate and/or complete, medical examination report using the (e.g., abbreviated) summary of the verbatim report, (e.g., all) relevant information and documentation of the medical examination can be (e.g., automatically, and/or in a timesaving manner) assembled and preserved in a comprehensive and concise form. Thereby, at minimal cost of electronic memory space, the accurate and/or complete medical examination report can be kept available for any future treatment, therapy planning, and/or consultation tailored to the patient. The electronic storage of the medical examination report further improves fast retrieval and transfer (e.g., securely, encrypted, and/or password protected), in particular across medical facilities, clinical sites, and/or medical specializations, e.g., facilitating holistic treatments. Thereby, the patient outcome can be improved long-term.

Alternatively, or in addition, by the technique, the conventional gap (and/or shortcomings) of an automated generation of clinical documentation, and/or medical examination reports by using smart communication technologies, such as smart speaker (and/or including a smart microphone), (e.g., video) camera and motion sensors (e.g., individually or collectively as the set of sensors) is bridged (and/or addressed), and novel, in particular multi-modal, abstractive meeting summarization techniques are leveraged. Further alternatively, or in addition, the technique can incorporate medical professional (e.g., physician) validation of the output of the acts of processing the received sensor data, generating the verbatim report, and/or converting the generated verbatim report into the medical examination report (collectively also denoted as meeting summarization algorithm), as well as visualization of the transcribed patient representative's (e.g., the patient's) utterances towards obtaining patient representative consent (in particular in line with privacy by design principles, e.g., including deleting all instances of data once the purpose for which it is collected has been accomplished, and/or a predetermined period of time has passed).

The visualization may be provided by a digital twin of the patient (also denoted as digital twin app of the patient, or briefly: digital twin app), e.g., running on a patient's electronic device (briefly: patient's device).

The technique is not limited to being applied to radiation oncology visits (as examples of medical examinations).

Alternatively, or in addition, the technique can be utilized in out-patient, and/or in-hospital, visit scenarios (as examples of medical examinations).

Alternatively, or in addition, the technique removes the limitations of conventional triple extraction using a multi-modal end-to-end trainable system, incorporates expert-in-the-loop (e.g., medical professional, in particular physician) verification of the reports, and/or removes potential privacy concerns of patients.

The medical examination may include a medical consultation. Alternatively, or in addition, the medical examination may include a multi-participant meeting.

The medical examination may take place in the medical professional's office (e.g., a doctor's office). E.g., the set of sensors may be deployed in the medical professional's office.

The medical professional may include a medical practitioner (also: physician, and/or medical doctor, briefly: doctor). Alternatively, or in addition, the medical professional may include an expert healthcare professional, and/or an assisting person (e.g., a medical assistant, physician assistant, nurse, and/or dental hygienist). Further alternatively, or in addition, the medical professional may include two or more persons, e.g., a medical practitioner and an assisting person.

The medical professional may be specialized in the field of the medical examination, e.g., as radiologist, (in particular radiological, and/or radiation) oncologist, cardiologist, neurologist, nephrologist, and/or endocrinologist.

The patient representative may include the patient (e.g., himself and/or herself). Alternatively, or in addition, the patient representative may include a person authorized to act on behalf of the patient (e.g., a parent, relative, carer, and/or caretaker), in particular in case the patient is underage and/or impaired. Further alternatively, or in addition, the patient representative may include two or more persons, e.g., the patient and one or more accompanying persons (e.g., including a person authorized to act on behalf of the patient).

The medical examination report may be part of, and/or may be included in, an electronic health record (EHR). The EHR may alternatively be denoted as electronic medical report (EMR). Alternatively, or in addition, the electronic medical report database may include the EHR, and/or EMR.

The set of sensors may include the at least one microphone and at least one further sensor. The at least one further sensor may include a (e.g., video) camera, and/or a motion sensor.

Alternatively, or in addition, the microphone may be integrated with the at least one further sensor, e.g., with the (in particular video) camera. Further alternatively, or in addition, the microphone may be included in a (e.g., voice activated, and/or smart) speaker.

Performing the method using two or more sensors may be denoted as multi-modal. E.g., the (in particular multi-modal) set of sensors may include a microphone and a (e.g., video) camera.

The sensor data may also be denoted as, in particular digital, recording.

Processing the audio data received from the microphone may include performing speech recognition, in particular by a speech recognition program (e.g., without identifying the speaker, in particular without identifying which parts of the audio data originate from the medical professional and which parts of the audio data originate from the patient representative). Alternatively, or in addition, the processing of the audio data received from the microphone may include identifying any one of the participants of the medical examination (e.g., the medical professional, and/or the patient representative), e.g., by a speech pattern, and/or a frequency pattern, of the participant.

Alternatively, or in addition, processing the received sensor data of the (e.g., video) camera (which may also be denoted as visual data) may include facial recognition, recognition of motions (also: movements), in particular of the lips, recognition of gestures, and/or any further identification of any one of the participants of the medical examination (e.g., the medical professional, and/or the patient representative). The further identification may include, e.g., identifying the medical professional according to an attributed position (e.g., a side of a desk in a doctor's office), and/or according to professional clothing. Alternatively, or in addition, the further identification may include, e.g., identifying the patient representative according to an attributed position (e.g., another side of a desk in a doctor's office, and/or an examination chair), and/or according to non-professional clothing (e.g., not wearing clothes resembling a medical work coat in shape and/or color).

Further alternatively, or in addition, processing the received sensor data of the motion sensor (which may include visual data) may include recognition of gestures of any participant of the medical examination (e.g., the medical professional, and/or the patient representative). Alternatively, or in addition, the processing of the motion sensor may include identifying any one of the participants of the medical examination (e.g., the medical professional, and/or the patient representative), e.g., according to their attributed position, and/or according to characteristic gestures (in particular performed by the medical professional).

The verbatim report may also be denoted as (e.g., verbal, verbatim, word-for-word, and/or literal) transcription and/or transcript.

Generating the verbatim report may include combining the received sensor data from the microphone, and/or from two or more microphones, and/or from the one or more further sensors. E.g., the generating of the verbatim report may include complementing (and/or combining) the audio data with visual data. The combining of the audio data with the visual data may include assigning each utterance, and/or text excerpt to the medical professional and/or to the patient representative.

The utterances may also be denoted as statements, pronouncements, expressions, and/or comments.

Converting the generated verbatim report into the medical examination report may include applying natural language processing (NLP) to the verbatim report. Alternatively, or in addition, converting the generated verbatim report into the medical examination report may be performed by an artificial intelligence (AI), neural network (NN), deep learning (DL), and/or reinforcement learning (RL).

The summary of the verbatim report may also be denoted as abstractive (and/or abbreviated) summary.

The (e.g., abstractive, and/or abbreviated) summary may be generated (e.g., separately, and/or independently) for the excerpts of the text assigned to the medical professional and to the patient representative. E.g., the conversion of the verbatim report may preserve the utterances by the patient representative word-by-word, and/or may convert (in particular abbreviate, and/or transform into, especially standardized, expert terminology) the utterances by the medical professional, in particular using NLP.

The predetermined ontology database may include a database of medical expressions and/or of expert terminology (e.g., words, multiword expressions, and/or phrases). Alternatively, or in addition, the predetermined ontology database may include (e.g., only, and/or may be reduced to) the medical expressions, and/or expert terminology, assigned to the medical specialization (e.g., radiology, and/or, in particular radiological, oncology) of the medical professional.

Storing the medical examination report in an electronic medical report database may include storing the medical examination report (e.g., centrally) at a medical facility (also: clinical site, clinic, hospital, and/or doctor's office). Alternatively, or in addition, storing the medical examination report in an electronic medical report database may include storing the medical examination report (e.g., locally) in a digital twin of the patient, and/or on a patient's device.

The set of sensors may include at least one further sensor, e.g., in addition to a microphone. The at least one further sensor may include a camera, in particular a video camera. Alternatively, or in addition, the at least one further sensor may include a motion sensor.

The method may further include an act of providing the text excerpts of the converted medical examination report, which are assigned to the patient representative, to the patient representative for approval. The method may still further include an act of receiving a user input by the patient representative. The user input may be indicative of approval, and/or rejection, in particular of the correctness, of the provided text excerpts assigned to the patient representative.

Any user input (e.g., by the patient representative, and/or by the medical professional) may be received via a (e.g., graphical) user interface (UI, in particular GUI).

The received user input (e.g., by the patient representative) may include an approval of a subset, and/or of the full set, of text excerpts assigned to the patient representative. Alternatively, or in addition, the received user input may include a rejection of a subset, and/or of the full set, of text excerpts assigned to the patient representative. The rejection may be indicative of the patient representative not consenting to digitally storing the corresponding text excerpts. Alternatively, or in addition, the rejection may be indicative of the patient representative assessing the corresponding text excerpts as incorrectly transformed into text (also: transcribed).

Alternatively, or in addition, the user input (e.g., by the patient representative, and/or by the medical professional) may include corrections to the provided text excerpts. E.g., the UI (and/or GUI) may include a (in particular text) editor for modifying the provided text excerpts, and/or for adding comments.

Alternatively, or in addition, comments may be selected based on predefined icons, and/or a predefined scale (e.g., indicating the correctness of the transcript).

The receiving of the user input by the patient representative may be time restricted. E.g., after a time period (also: lapse of time), e.g., two days, without user input responsive to the provided text excerpts, the provided text excerpts may be classified as approved.

Alternatively, or in addition, the method may further include an act of providing the text excerpts of the converted medical examination report, which are assigned to the medical professional, to the medical professional for verification. The method may still further include an act of receiving a user input, by the medical professional, indicative of a validation of the provided text excerpts assigned to the medical professional.

The providing of the text excerpts of the converted medical examination report to the medical professional may include providing the full converted medical examination report (in particular also including the text excerpts assigned to the patient representative) to the medical professional. The providing of the full converted medical examination report may be subject to the approval of the patient representative (e.g., by the act of receiving the user input by the patient representative).

The user input by the medical professional may include corrections, additions, and/or one or more (e.g., manual) annotations.

The method may further include an act of temporarily storing intermediate data in a private cloud. The intermediate data may include the received sensor data, the generated verbatim report, and/or at least parts of the converted medical examination report. The method may still further include an act of deleting the temporarily stored intermediate data from the private cloud after a predetermined period of time, after a rejection by the patient representative, and/or after a verification by the medical professional.

The private cloud may also be denoted as private access cloud (PAC), and/or as protected cloud. The protection may include a password, an encryption, and/or a firewall. Alternatively, or in addition, the private cloud may serve for privacy protection of (e.g., sensitive) patient data.

The temporarily storing may also be denoted as intermediate storing.

Storing the medical examination report in an electronic medical report database (e.g., in the long term) may include storing the medical examination report in a centralized memory of a medical facility, in particular of the medical facility where the medical examination was performed.

Alternatively, or in addition, a copy of the medical examination report may be sent (e.g., securely) from the (e.g., specialized) medical professional to a general practitioner (also: family doctor) and stored in a (e.g., centralized) memory there. Further alternatively, or in addition, the medical examination report may be sent, e.g., upon request, to another medical professional of the same, and/or a different, medical field of the medical examination.

The method may further include an act of outputting the stored medical examination report.

Outputting the stored medical examination report may include retrieving the medical examination report from the electronic medical report database, e.g., for display and/or information at a later consultation.

Alternatively, or in addition, storing the medical examination report in an electronic medical report database may include storing the medical examination report in a digital twin of the patient. Alternatively, or in addition, the providing of the text excerpts assigned to the patient representative and the receiving of the user input by the patient representative may be performed using the digital twin of the patient.

The digital twin of the patient may include an interface, memory, and/or database, for storing and/or viewing by the patient representative, health-related metrics, and/or documents of the patient. Alternatively, or in addition, the digital twin may include and/or store, a patient health record. The patient health record may include the patient's daily activities (e.g., sleep, exercise, and/or diet), the patient's medical, and/or surgical, history, the patient's vital signs (e.g., received from a wearable device), the patient's laboratory (short: lab) test results, and/or the patient's medical imaging data (briefly: medical images).

Alternatively, or in addition, the digital twin may include the text excerpts of the converted medical examination report, and/or of the verbatim report of the medical examination, assigned to the patient representative. Approving (also: consenting) or rejecting (also: refusing) the text excerpts assigned to the patient representative may be performed by the digital twin.

The text excerpts of the converted medical examination report assigned to the patient representative may only be forwarded to, and/or provided to, the medical professional upon approval (also: consent) of the patient representative.

The assignment of each excerpt of the text to the medical professional or the patient representative in the act of generating a verbatim report may be based on filtering of the audio data according to a frequency pattern, and/or according to a low pass filter, and/or a high pass filter. The filtered audio data may be classified according to characteristics determined by the filtering. The classifying characteristics may be assigned to the medical professional and/or the patient representative.

Alternatively, or in addition, the assignment of each excerpt of the text to the medical professional or the patient representative in the act of generating a verbatim report may be based on extracting time stamps from the audio data and from the further sensor data, combining the audio data and the further sensor data, in particular visual data, according to the extracted time stamps, and assigning each excerpt of the text to the medical professional or the patient representative according to the combination of the audio data and the further sensor data, in particular the visual data, per time stamp.

The method may further include an act of identifying, based on the processed sensor data, medical imaging data that were examined during the medical examination. Alternatively, or in addition, the method may further include an act of appending the medical imaging data to the medical examination report.

The medical imaging data may be obtained (e.g., prior to the medical examination) by medical imaging. Alternatively, or in addition, the medical imaging data may be obtained in relation to the patient, who is the subject of the medical examination.

The medical imaging may include a computed tomography (CT) scan, a magnetic resonance imaging (MRI) scan, an ultrasound (US) scan, a positron emission tomography (PET) scan, a single photon emission computed tomography (SPECT) scan, and/or an X-ray scan (also denoted as radiography scan). Alternatively, or in addition, the medical imaging data may be stored in a picture archiving and communication system (PACS), e.g., at the medical facility, clinical site, and/or location, of the medical professional's office.

Examining the medical imaging data may include viewing, and/or discussing, the medical imaging data, in particular during the medical examination.

The medical imaging data may be appended to, and/or included in, the medical examination report. Alternatively, or in addition, appending the medical imaging data to the medical examination report may include including a link to a storage location of the medical imaging data, e.g., within a medical facility and/or clinical site.

Alternatively, or in addition, the medical professional may correct the identification of, and/or may add, medical imaging data to be appended to the medical examination report.

Converting the generated verbatim report into a medical examination report may be based on a trained artificial intelligence (AI), a trained neural network (NN), deep learning (DL), and/or reinforcement learning (RL).

The NN may include a bidirectional autoregressive transformer (BART) and/or at least one decoder, preferably one decoder per sensor.

Alternatively, or in addition, the AI, NN, DL, and/or RL may be based on NLP, entity recognition (in particular of medical terminology), and/or entity linking (in particular of medical terminology) according to the ontology database. Further alternatively, or in addition, the AI, NN, DL, and/or RL may be based on mapping sequences (also denoted as multiword expressions, and/or phrases) to medical concepts, and/or medical terminology included in the ontology database.

The training of the AI and/or the NN, and/or the learning of the DL and/or the RL, may be based on training data sets. Each training data set may include sensor data and an associated, in particular at least manually compiled, medical examination report.

The (e.g., at least manually compiled) medical examination report within the training data set may be denoted as ground truth. The ground truth may be provided by a medical professional.

The training, and/or learning, may be based on optimizing a loss function.

The loss function may include a reconstruction loss. Optimizing the loss function may include minimizing the reconstruction loss.

Alternatively, or in addition, the loss function may include a reward. Optimizing the loss function may include maximizing the reward. Alternatively, or in addition, the reward may be based on an n-gram overlap of the output, by the AI, NN, DL, and/or RL, of a medical examination report with the ground truth (e.g., the at least manually compiled medical examination report).

The training, and/or learning, may be unsupervised, and/or supervised.

The medical examination may include a radiology, and/or an, in particular radiological, oncology examination.

As to a device aspect, a computing device (computer) for generating a medical examination report from sensor data of a medical examination is provided. The computing device includes a sensor interface configured to receive sensor data from a set of sensors in relation to a medical examination. The set of sensors includes at least one microphone. The sensor data include at least audio data (e.g., captured by the at least one microphone). The audio data include utterances by a medical professional and a patient representative participating in the medical examination.

The computing device further includes a processing module configured to process the received sensor data according to the type of sensors. The processing includes at least transforming the audio data into text. The computing device further includes a generating module configured to generate a verbatim report of the medical examination based on the processed sensor data. The verbatim report includes each excerpt of the text being assigned to the medical professional or the patient representative. The computing device further includes a converting module configured to converting the generated verbatim report into a medical examination report. The medical examination report includes a summary of the verbatim report. The summary includes vocabulary, and/or text excerpts, by accessing a predetermined ontology database in relation to a medical field of the medical examination. The computing device still further includes a storage interface configured to forward the medical examination report to an electronic medical report database (memory) for storing.

Any of the above modules of the computing device may also be denoted as unit. Alternatively, or in addition, any combination of the processing module, the generating module, and/or the converting module may be included in or be implemented by a (in particular centralized) processing unit (e.g., a CPU). The processing unit may also briefly be denoted as processor.

As to a system aspect, a (e.g., distributed) system for generating a medical examination report from sensor data of a medical examination is provided. The (e.g., distributed) system includes a set of sensors including at least one microphone. The set of sensors is configured to capture sensor data relating to a medical professional and a patient representative participating in the medical examination. The sensor data include at least audio data (e.g., captured by the at least one microphone). The audio data include utterances by the medical professional and the patient representative.

The (e.g., distributed) system further includes a processing module configured to process the sensor data according to the type of sensors. The processing includes at least transforming the audio data into text. The (e.g., distributed) system further includes a generating module configured to generate a verbatim report of the medical examination based on the processed sensor data. The verbatim report includes each excerpt of the text being assigned to the medical professional or the patient representative. The (e.g., distributed) system further includes a converting module configured to convert the generated verbatim report into a medical examination report. The medical examination report includes a summary of the verbatim report. The summary includes vocabulary, and/or text excerpts. The (e.g., distributed) system further includes an ontology database in relation to a medical field of the medical examination. The ontology database includes the vocabulary, and/or the text excerpts for the summary of the verbatim report. The ontology database is configured to be accessed for converting the generated verbatim report into the medical examination report. The (e.g., distributed) system still further includes a storage module configured to store the medical examination report in an electronic medical report database.

Any of the above modules of the (e.g., distributed) system may also be denoted as unit. Alternatively, or in addition, any combination of the processing module, the generating module, and/or the converting module may be included in or implemented by a (in particular centralized) processing unit (e.g., a CPU). The processing unit may also briefly be denoted as processor.

The (e.g., distributed) system may at least partially be embodied by a computing cloud (briefly: cloud). Alternatively, or in addition, the processing module, the generating module, the converting module, the ontology database, and/or the electronic medical report database (and/or the storage module configured to store the medical examination report in an electronic medical report database) may be part of a computing cloud.

The (e.g., computing) cloud may be a private, and/or protected, cloud. Alternatively, or in addition, the protection of the (e.g., computing) cloud (briefly: cloud protection) may include a password protection, firewall, and/or an access restriction (e.g., access reserved to verified devices, and/or to verified users).

By the (e.g., computing) cloud being private, and/or protected, sensor data (in particular related to the patient) may be protected.

The system may be distributed in the sense that the hardware, on which the modules and/or the interfaces are implemented, may be distributed, and/or may include two or more physical entities (e.g., processors).

As to a further aspect, a computer program including program elements which, when the program elements are loaded into a memory of a computing device (and/or a, e.g., distributed, system), induce the computing device (and/or the, e.g., distributed, system) to carry out the acts of the method for generating a medical examination report from sensor data of a medical examination according to the method aspect is provided.

As to a still further aspect, a non-transitory computer-readable medium, on which program elements are stored that can be read and executed by a computing device (and/or a, e.g., distributed, system), is provided in order to perform acts of the method, when the program elements are executed by the computing device (and/or by the, e.g., distributed, system), according to the method aspect for generating a medical examination report from sensor data of a medical examination is provided.

The properties, features and advantages described above, as well as the manner they are achieved, become clearer and more understandable in the light of the following description and embodiments, which will be described in more detail in the context of the drawings. This following description does not limit the invention on the contained embodiments. Same components or parts can be labelled with the same reference signs in different figures. In general, the figures are not for scale.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 is a flow chart of a method for generating a medical examination report from sensor data of a medical examination according to a preferred embodiment;

FIG. 2 is an overview of the structure and architecture of a computing device for generating a medical examination report from sensor data of a medical examination according to a preferred embodiment, wherein the computing device may be configured to perform the method of FIG. 1;

FIG. 3 is an overview of the structure and architecture of a, in particular distributed, system for generating a medical examination report from sensor data of a medical examination according to another preferred embodiment, in which method acts, e.g., according to the method of FIG. 1 are shown as well;

FIG. 4 is a schematic overview of an example neural network architecture for processing (e.g., individually per sensor) sensor data, generating a verbatim report (e.g., collectively based on the sensor data from all sensors), and converting the verbatim report into the medical examination report, in particular according to the method of FIG. 1, wherein the neural network architecture may be included in the computing device of FIG. 2 and/or the, in particular distributed, system of FIG. 3; and

FIG. 5 is an exemplary illustration of a subjective-objective-assessment-plan (SOAP) section and/or subsection masking, which may be used when performing the method of FIG. 1.

### DETAILED DESCRIPTION

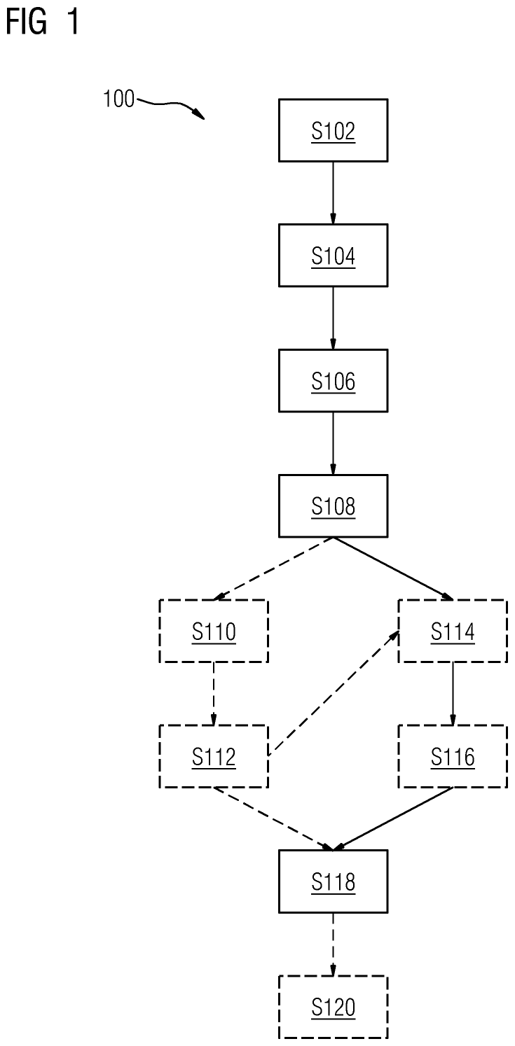

FIG. 1 schematically illustrates a computer implemented method for generating a medical examination report from sensor data of a medical examination. The method is generally denoted by the reference sign 100.

The method 100 includes an act S102 of receiving sensor data from a set of sensors in relation to a medical examination. The set of sensors includes at least one microphone. The sensor data include at least audio data (in particular captured by the at least one microphone). The audio data include utterances by a medical professional and a patient representative participating in the medical examination.

The method 100 further includes an act S104 of processing the received S102 sensor data according to the type of sensors. The processing S104 includes at least transforming the audio data into text.

The method 100 further includes an act S106 of generating a verbatim report of the medical examination based on the processed S104 sensor data. The verbatim report includes each excerpt of the text being assigned to the medical professional or the patient representative.

The method 100 further includes an act S108 of converting the generated S106 verbatim report into a medical examination report. The medical examination report includes an (e.g., abbreviated) summary of the verbatim report. The summary includes vocabulary, and/or text excerpts, obtained (and/or received, and/or retrieved) by accessing a predetermined ontology database in relation to a medical field of the medical examination.

The method 100 still further includes an act S118 of storing the medical examination report in an electronic medical report database.

Optionally, the method 100 includes an act S110 of providing the text excerpts of the converted S108 medical examination report, which are assigned to the patient representative, to the patient representative for approval. The method 100 may further include an act S112 of receiving a user input by the patient representative. The user input may be indicative of approval, and/or rejection, in particular of the correctness, of the provided $110 text excerpts assigned to the patient representative.

Further optionally, the method 100 includes an act S114 of providing the text excerpts of the converted S108 medical examination report, which are assigned to the medical professional, to the medical professional for verification. The method 100 may further include an act S116 of receiving a user input, by the medical professional, indicative of a validation of the provided S114 text excerpts assigned to the medical professional.

Still further optionally, the method 100 includes an act S120 of outputting the stored S118 medical examination report.

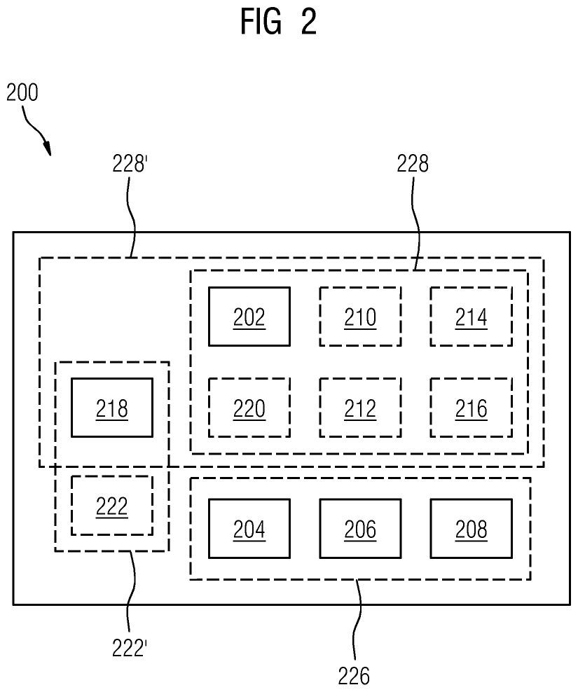

FIG. 2 schematically illustrates a computing device for generating a medical examination report from sensor data of a medical examination. The computing device is generally referred to by the reference sign 200.

The computing device 200 includes a sensor interface 202 configured to receive sensor data from a set of sensors in relation to a medical examination. The set of sensors includes at least one microphone. The sensor data include at least audio data (e.g., captured by the at least one microphone). The audio data include utterances by a medical professional and a patient representative participating in the medical examination.

The computing device 200 further includes a processing module 204 configured to process the received sensor data according to the type of sensors. The processing includes at least transforming the audio data into text.

The computing device 200 further includes a generating module 206 configured to generate a verbatim report of the medical examination based on the processed sensor data. The verbatim report includes each excerpt of the text being assigned to the medical professional or the patient representative.

The computing device 200 further includes a converting module 208 configured to convert the generated verbatim report into a medical examination report. The medical examination report includes a summary of the verbatim report. The summary includes vocabulary, and/or text excerpts, by accessing a predetermined ontology database in relation to a medical field of the medical examination.

The computing device 200 still further includes a storage interface 218 configured to forward the medical examination report to an electronic medical report database for storing.

Optionally, the computing device 200 includes a first text excerpts sending interface 210 configured for providing the text excerpts of the converted medical examination report, which are assigned to the patient representative, to the patient representative for approval. Alternatively, or in addition, the computing device 200 may include a first user input receiving interface 212 for receiving a user input by the patient representative. The user input may be indicative of approval, and/or rejection, in particular of the correctness, of the provided text excerpts assigned to the patient representative.

Further optionally, the computing device 200 includes a second text excerpts sending interface 214 configured for providing the text excerpts of the converted medical examination report, which are assigned to the medical professional, to the medical professional for verification. Alternatively, or in addition, the computing device 200 may include a second user input receiving interface 216 for receiving a user input, by the medical professional, indicative of a validation of the provided text excerpts assigned to the medical professional.

Still further optionally, the computing device 200 includes an output interface 220 for outputting the stored medical examination report.

Any of the above modules 204; 206; 208 may also be denoted as unit.

Alternatively, or in addition, any (e.g., pairwise, and/or overall) combination of the processing module 204, the generating module 206, and/or the converting module 208 may be included in or implemented by a (in particular centralized) processing unit 226 (e.g., a CPU). The processing unit 226 may also briefly be denoted as processor 226.

Further alternatively, or in addition, any one of the interfaces 202; 210; 212; 214; 216; 218 may be included in an external interface 228′. Alternatively, or in addition, the external interface 228 may, e.g., include the interfaces 202; 210; 212; 214; 216.

Still further alternatively, or in addition, the storage interface 218 may include an internal interface to a (e.g., internal) memory 222 including the electronic medical report database. The internal storage interface 218 and memory 222 may be collectively denoted as (e.g., internal) memory 222′.

Alternatively, or in addition, the first text excerpts sending interface 210 and the first receiving user input interface 212 may be included in a patient representative interface.

Alternatively, or in addition, the second text excerpts sending interface 214 and the second receiving user input interface 216 may be included in a medical professional interface.

Further alternatively, or in addition, the first text excerpts sending interface 210, the first receiving user input interface 212, the second text excerpts sending interface 214 and the second receiving user input interface 216 may be included in a user interface.

The computing device 200 may be configured to perform the method 100.

Alternatively, or in addition, a (e.g., distributed) system may include any one of the modules 204; 206; 208, interfaces 202; 210; 212; 214; 216; 218; 220; 228; 228′, memory 222; 222′, and/or processing unit 226 disclosed in the context of the computing device 200. The (e.g., distributed) system may generally be denoted by reference sign 300.

Further alternatively, or in addition, the (e.g., distributed) system 300 may be cloud-based, and/or may include a (e.g., private) cloud.

The (e.g., distributed) system 300 may be configured to perform the method 100.

The technique (e.g., including the method 100, computing device 200, and/or system 300) focuses on addressing the gap of automated generation of clinical documentation (in particular medical examination reports) by using smart communication technologies, such as smart speaker (and/or microphone), camera and motion sensors (independently or collectively as the set of sensors), and novel (in particular multi-modal) abstractive meeting summarization techniques. Alternatively, or in addition, the technique incorporates medical professional (e.g., physician) validation of the output of a meeting summarization algorithm (e.g., including the acts of processing S104 the received S102 sensor data, of generating S106 a verbatim report of the medical examination based on the processed S104 sensor data, and/or of converting S108 the generated S106 verbatim report into the medical examination report). Further alternatively, or in addition, the technique can provide a visualization of the transcribed patient representative's (e.g., the patient's) utterances towards obtaining patient representative consent (in particular in line with privacy by design principles).

The technique can be applied to radiation oncology visits (as examples of medical examinations). Alternatively, or in addition, the technique can be utilized in out-patient, and/or in-hospital, visit scenarios (as examples of medical examinations).

The technique can remove limitations of conventional triple extraction using a (in particular multi-modal) end-to-end trainable system (e.g., including an AI, NN, DL, and/or RL). Alternatively, or in addition, the technique can incorporate expert-in-the-loop (e.g., medical professional, in particular physician) verification of the medical examination reports, and/or of the clinical documentation. Alternatively, or in addition, the technique can disperse, extinguish, and/or obviate potential privacy concerns of patient representatives (e.g., patients).

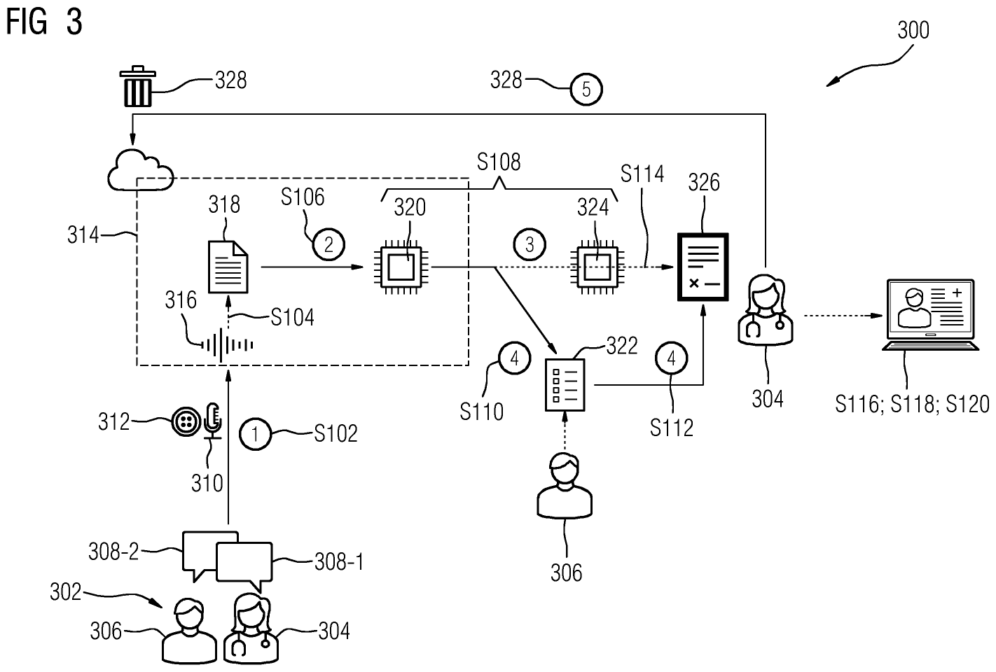

FIG. 3 shows an exemplary embodiment of the (e.g., distributed) system 300. The system 300 in the example of FIG. 3 may alternatively be denoted as (e.g., radiation oncology) automated documentation system 300.

As shown in FIG. 3, an embodiment of the technique involves using smart communication technologies (e.g., a smart speaker and/or microphone 310, and/or a further sensor 312, in particular a video camera 312, shortly also denoted as camera 312) during a medical examination 302 (also denoted as follow-up patient visit), e.g., for radiation oncology and/or radiotherapy.

The dialogue between the radiation oncologist (as embodiment of the medical professional 304) and patient (and/or including relatives of the patient present, as patient representative 302) including utterances 308-1 by the medical professional 304 and utterances 308-2 by the patient representative 306 are captured in the act S102 (e.g., by the microphone 310 as audio files schematically depicted at reference sign 316 in FIG. 3, and potentially as non-verbal data captured by one or more cameras 312 and/or, in particular motion, sensors 312) and sent to a cloud 314 storage.

In the particular embodiment of FIG. 3, no local (e.g., in the doctor's office) copy of the files (in particular including the sensor data 316) is persisted. The audio file 316 is translated S104 to a verbatim report (also: text file) 318 using a speech-to-text application programming interface (API) provided, (and/or offered) by the cloud 314 platform provider (e.g., including Microsoft azure cognitive services). The verbatim report (also: text file) 318 (and optionally, in particular a transcript and/or evaluation S106 of, the non-verbal data) are then sent as an input into a cloud-based deployment of a multi-modal meeting summarization model 320; 324.

In one embodiment, a state-of-the-art approach for multi-modal meeting (as the medical examination 302 with multiple sensors 310; 312 recording sensor data 316) summarization 320 is used, with an additional task of standardization 324 (e.g., corresponding to, and/or including, the act S108 of converting the verbatim report into the medical examination report), e.g., denoted as summarize-normalize paradigm. The multi-modal summarization model 320 (e.g., including, and/or correspond to, the method acts of processing S104 the sensor data, generating S106 the verbatim report, and/or converting the verbatim report 318 into the medical examination report) according to an embodiment is trained on millions of video frames and associated captions (also denoted as text excerpts and/or annotations, and/or including verbatim reports 318 and/or medical examination reports associated with the video frames) that include, which speaker (e.g., the medical professional 304 and/or the patient representative 302) said what 308-1; 308.

In an alternative embodiment, the act S108 of converting the verbatim report into the medical examination report (and/or the additional task of standardization 324) may affect, and/or concern, both the verbatim report of the utterances 308-1 of the medical professional 304 and the utterances 308-2 of the patient representative 308-2.

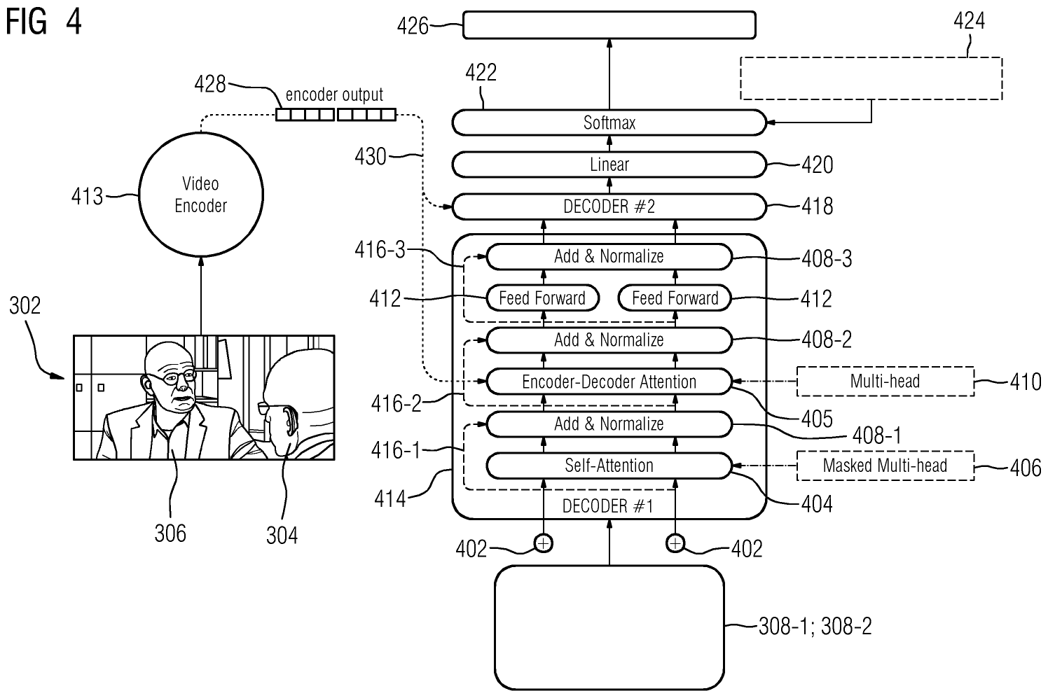

FIG. 4 exemplarily illustrates a neural network (NN) architecture for processing S104 the received S102 sensor data, generating S106 the verbatim report, and/or converting S108 the generated S106 verbatim report into the medical examination report. The NN architecture of FIG. 4 may also be denoted as multi-modal query-focused summarization architecture.

As exemplarily illustrated in FIG. 4, the video frames of the medical examination 302 are passed as input into a video encoder 413 that represents the pixels with vectors signifying the objects and persons in the clips. The encoded output 428 is passed 430 to various layers 405 of a decoder 414; 418 that include a vector representation of the associated caption for each video clip (e.g., including the video frames).

Alternatively, or in addition, an input embedding 402 may refer to a vector representation of a token, e.g., word embeddings.

At reference sign 402, positional embeddings, e.g., a vector representation of the absolute and/or relative position of a token in a sequence of tokens, is input into the first decoder 414 and/or the second decoder 418. The positional embeddings 402 are combined with the input embeddings to the first decoder 414 and/or the second decoder 418, e.g., vector representation of the text-based utterances 308-1; 308-2.

Herein, a token may include a word, and/or a sequence of (in particular consecutive) words, of sensor data captured by a microphone 310. Alternatively, or in addition, one or more (e.g., collection and/or accumulation of) pixels may include a gesture, and/or lip movements, captured by a further sensor, in particular a camera 312, and/or a motion sensor 312.

The positional embeddings may, e.g., correspond to, and/or may include, time stamps in relation to utterances 308-1; 308-2, gestures, and/or lip movement.

The self-attention sublayer 404 in the decoder 414 (and/or of the second decoder 418) of the embodiment of the NN architecture in FIG. 4 takes as input 402 a sequence of tokens (and/or representing captions, wherein a caption may refer to, and/or may include, a text excerpt), h₁, . . . , hₙ (also denoted as, in particular sequence, of vectors, and/or as vector representation). Each vector is linearly transformed into the query, key and value vectors denoted by the symbols qᵢ, kᵢ and vᵢ, respectively. All the vectors qᵢ, kᵢ and vᵢ are packed into the Q, K and V matrices, respectively. The matrices Q and K are initialized with random weights, W_{Q} and W_{K}, respectively. A SoftMax function (also denoted as SoftArgMax, and/or normalized exponential function, e.g., functioning in order to convert a matrix into a probability distribution, in particular of possible outcomes) is applied to the product of Q and (in particular the transposed of) K (e.g., scaled using the square root of the dimension of K) to calculate the weight for V, i.e., W_{V}. The attention matrix for the sublayer 404 (Attention (Q, K, V)) is the summation of V (W_{V}) from all possible combination pairs of vectors from Q and K, as displayed in Eq. (1):

$$Attention ( Q , K , V ) = softmax ( \frac{Q \cdot {K}^{T}}{\sqrt{{d}_{k}}} ) V \qquad ( 1 )$$

In the architecture of FIG. 4, the ‘add’ sublayer 408-1; 408-2; 408-3 represents a residual connection between the self-attention sublayers 404 across different blocks (408-1), and between the self-attention sublayer 404 and the simple position-wise fully connected feed forward network 412 within a block. These connections force the input of the subsequent sublayer 405; 412; 418 to include the original token vectors (h₁, . . . , hₙ) alongside the output of the previous sublayer 408-1; 408-2; 408-3 (e.g., decoded pixels, in particular of a camera 312 encoding 413, and tokens). The ‘normalize’ aspect (and/or performing the act S108 of converting the verbatim report 318 into the medical examination report) may represent a function to transform all outputs from the self-attention layers 404 into a specific dimension (e.g., d_{model}=512).

The decoder 414 and/or the second decoder 418 in the NN architecture embodiment of FIG. 4 also includes the encoder-decoder attention sublayer 405. The sublayer 405 essentially functions the same way the self-attention sublayer 404 does, except that the query matrix Q is derived from the input vectors of the decoder 414; 418 (e.g., tokens from captions), while the key and value matrices (K and V, respectively) are derived from the encoder output 428 (e.g., pixels from videos, e.g., captured by a camera 312). In other words, the sublayer 405 allows the tokens to ‘attend’ to the pixels, unlike the self-attention sublayer 404, in which the tokens ‘attend’ only to themselves. In turn, there is an additional add and normalize sublayer 408-2; 408-3 that represents a residual connection between the self-attention 404 and the encoder-decoder attention sublayers 405.

The input (and/or the input embedding 402), and/or any one of the add and normalize sublayers 408-1; 408-2; 408-3 may be connected, e.g., directly, by (e.g., skip and/or shortcut) connections 416-1; 416-2; 416-3. E.g., in addition to receiving the output from the self-attention sublayer 404, the add and normalize sublayer 408-1 may, in particular directly, receive the input embedding 402 through the connection 416-1. Alternatively, or in addition, a later add and normalize sublayer 408-2; 408-3 may, in addition to the output of the directly preceding sublayer 405; 412, receive the output from the preceding add and normalize sublayer 408-1; 408-2 through the corresponding connection 416-2; 416-3.

The self-attention sublayer 404 may include, and/or may be supplied by, a masked multi-head 406. Alternatively, or in addition, the encoder-decoder attention sublayer 405 may include, and/or may be supplied by, a multi-head 406.

The final output from the decoder (e.g., the first decoder 414, and/or the second decoder 418) in the NN architecture embodiment of FIG. 4 is pushed to a SoftMax layer 422, via a linear sublayer 420. The SoftMax layer 422 predicts a response 426 to an input query 424 by retrieving a span of tokens (that in particular include the answer), the input video (e.g., captured by the camera 312 of FIG. 3), and captions 308-1; 308-2 (e.g., captured by the microphone 310 of FIG. 3). In this way a, summary 426 (and/or medical examination report) of a conversation 308-1; 308-2 from the input video (in particular including audio data 316) can be provided.

The second decoder 418 in the NN architecture embodiment of FIG. 4 may be an, e.g., exact, replica of the first decoder 414 shown in greater detail in FIG. 4.

The resulting model (e.g., including, and/or corresponding to the trained, and/or learned, NN, AI, DL, and/or RL) for query-focused summarization 426 (and/or medical examination report generation), when streamlined to the exemplary use case of a medical examination report (in particular a radiation oncology documentation), focuses on generating a concise representation of a document (e.g., the verbatim report 318) based on a specific input query (see Zhu, H., Dong, L., Wei, F., Qin, B. and Liu, T., 2022. Transforming wikipedia into augmented data for query-focused summarization. IEEE/ACM Transactions on Audio, Speech, and Language Processing). E.g., ‘assessment’ may denote the query needed to generate the medical examination report, in particular the summary of the radiation oncologist's evaluation and/or diagnosis.

It is noted that Zhu, H., Dong, L., Wei, F., Qin, B. and Liu, T., 2022. Transforming wikipedia into augmented data for query-focused summarization. IEEE/ACM Transactions on Audio, Speech, and Language Processing, only focuses on extracting and/or retrieving Wikipedia sentences to include a summary based on a query, whereas the technique hwewin involves abstracting and/or generating a medical examination report (e.g., using concise, and/or standard, report language of the medical field of the medical examination) based on a query.

The summarize-normalize paradigm (e.g., embodied by the converting module 208, and/or assigned for performing the act S108 of converting the generated S106 verbatim report into the medical examination report) according to an embodiment includes generating an abstractive summary 326 (e.g., denoted as ‘summarize’, and/or as summary of the medical professional's text excerpts) from a medical professional 304 (e.g., a radiation oncologist's) utterances 308-1 related to the query, and then summarizing all other utterances 308-2 as the patient utterance summary 322 (also denoted as the patient representative's text excerpts). Alternatively, or in addition, the summarize-normalize paradigm according to an embodiment includes mapping the clinically relevant sentences and concepts in the medical professional's 304 (e.g., radiation oncologist's) summary to standard clinical vocabulary (e.g., in the act S108 of converting the generated S106 verbatim report into the medical examination report).

The utterances 308-1; 308-2 from various medial examinations 302 (also denoted as patient-physician encounters) can be manually annotated and linked to specific sections and/or subsections in a subjective-objective-assessment-plan (SOAP) visit note, representing the medical examination report (also denotes as the document and/or summary), e.g., of a radiation oncology follow-up visit as an example of a medical examination 302.

For locate and summarize (e.g., as the acts of processing S104 the received S102 sensor data, generating S106 the verbatim report, and/or converting S108 the generated S106 verbatim report into the medical examination report), a sequence-to-sequence (seq2seq) language model (LM) (e.g., including bidirectional autoregressive transformers (BART)) may be pretrained. The pre-training may be accomplished in a multi-modal setting (and/or using multiple sensors, in particular of at least two different types, and more particularly including a microphone 310 and a camera 312) with utterances-SOAP section and/or subsection as input alongside pixels and sensor time series which may represent additional vectors qᵢ and kᵢ used to train the LM.

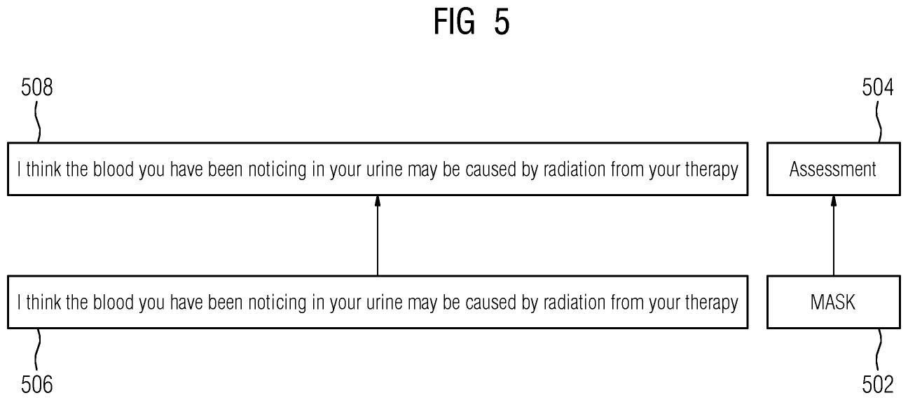

Alternatively, or in addition, a novel addition to the existing pre-trained seq2seq LM self-supervised objectives (e.g., token masking, token deletion, and/or sentence permutation) may be query masking, as exemplified in FIG. 5.

The LM, and/or the model (e.g., including, and/or corresponding to the trained, and/or learned, NN, AI, DL, and/or RL), according to an embodiment learns to predict the masked query 502 (and/or the assessment 504 thereof, e.g., at reference sign 508) in an input utterance-SOAP section/subsection pair 506 (and/or the mask 502), e.g., by minimizing a mask language modeling loss.

The pre-trained multi-modal seq2seq LM (mSeq2SeqPLM) may, according to an embodiment, be fine-tuned on a subset of medical examination 302 (e.g., radiation oncology) utterance-SOAP section/subsection data towards generating a preliminary medical examination report (also denoted as preliminary summary) focused mainly on the medical professional's 304 (e.g., radiation oncologist's) utterances 308-1 linked to SOAP sections/subsections.

According to some embodiments, the technique may include post-processing of the medical examination reports (also denoted as meeting summaries). E.g., the post-processing may include the act of generating S108 the medical examination report from the verbatim report 318.

In an embodiment, each sentence in the preliminary medical examination report (also denoted as preliminary summary) may be given (and/or provided) as an input to a model for medical entity linking, e.g., Medlinker (see Loureiro, D. and Jorge, A. M., 2020 April. Medlinker: Medical entity linking with neural representations and dictionary matching. In European Conference on Information Retrieval (pp. 230-237). Springer, Cham). MedLinker is trained by finetuning a large language model with the MedMentions dataset (see Sunil Mohan, Rico Angell, Nicholas Monath, Andrew McCallum. 2021. Low Resource Recognition and Linking of Biomedical Concepts from a Large Ontology. In Proceedings of the ACM Conference on Bioinformatics, Computational Biology, and Health Informatics (ACM-BCB), 2021). The MedMentions dataset contains 4392 randomly selected PubMed papers and is annotated with mentions of the unified medical language system (UMLS) entities. A little over 350000 entities are linked to concepts of 21 selected semantic types in UMLS. The MedLinker architecture uses a named entity recognition (NER) system producing mentions that are matched to entities using independent approaches based on n-grams and contextual embeddings, which are combined in a post-processing act into the final entity predictions. The named entity recognition system may include a conventional NER finetuning, where the last states of the language model are pooled and bidirectional long-short term memory-conditional random field (BiLSTM-CRF) may be applied on the pooled representation to generate entity spans. MedLinker uses three different types of matching algorithms to identify the final UMLS concept: firstly a zero-shot linking with approximate dictionary matching using 3-gram SimString matching, secondly linking by similarity to entity embeddings where the UMLS concepts are encoded using their concept and the last four layers are pooled, and thirdly training a minimal SoftMax classifier using contextual embeddings from large language models.

There exist several other entity linking systems that are trained on other corpus like BioCreative V CDR (BC5CDR), National Center for Biotechnology Information (NCBI), Cometa, and AskAPatient. Notable systems include generative entity linking systems like GENRE, which uses generative language models to disambiguate entity mentions by auto-regressively generating the standard concept names conditioned on the inputs, GenBioEl, which achieves state-of-the-art entity linking performance on various biomedical entity linking datasets by generative methods, and BioBART-based entity linkers which have state-of-the-art performance on finetuning on entity linking datasets.

The entity linking model (e.g., embodied by the converting module 208, and/or assigned for performing the act S108 of converting the generated S106 verbatim report into the medical examination report) may be modified according to the technique towards mapping sequences (e.g., beyond individual entities) to appropriate medical concepts (e.g., blood in the urine caused by radiotherapy may be normalized to radiation-induced hematuria). The entity linking model may leverage standard clinical ontologies like ICD10 and SNOMED-CT, such that the preliminary examination report (also denoted as preliminary summary) can be easily used for reporting of quality measures, and/or for use in future medical examinations 302 in the same, and/or a different (in particular, a related), medical field.

In free flow conversations (e.g., as typical for medical examinations 302), entity linking (e.g., performing the act S108 of converting the generated S106 verbatim report into the medical examination report) is a hard problem since it is difficult to identify the right context. For example, medical examinations 302 (also denoted as physician-patient conversations) may not, or need not, indicate specific areas of localization of certain symptoms, especially when the patient representative 306 (e.g., the patient) describes the symptoms ambiguously, and/or only indicated the localization by gestured.

Alternatively, or in addition, there can be ambiguous spans of entities where the patient representative 306 (e.g., the patient) might be referring to multiple locations, and/or might be using (e.g., more) colloquial terms for the entities—The system 300 (and/or the device 200) may fail to understand the spans of the relevant entities.

Deep reinforcement learning (DRL)-based entity recognition and/or linking can become useful, e.g., on even a global scale, where the system 300 (and/or the computing device 200) learns to understand spans of several medical entities and/or the exact concept they are referring to. For each decoding act, the DRL algorithm may determine (e.g., compute) an utterance-level reward by comparing entities in the generated utterance (e.g., from the seq2seq LM) and the ground-truth entities (e.g., in the training data and/or training utterance) as shown in Eq. (2):

$$r = ROGUE ( \{ {E}_{U} \} , {E}_{o} ) \qquad ( 2 )$$

$$a = π ( r )$$

In Eq. (2), r is the reward, ROUGE (recall-oriented understudy for gisting evaluation) refers to the n-gram overlaps of tokens and/or words between entities in the generated output (E) and those in the ground truth (Eₒ), a is the action that should be taken (acceptance and/or rejection, e.g., by the entity linking model, of the generated entity), and n is the policy network on which the actions are based.

In another embodiment, a medical examination report 302 (also denoted as summary) of patient representative 306 (e.g., patient) utterances 308-2 may be generated. Due to the fact that the subjective sections and/or subsections conventionally include patient narratives, such utterances 308-2 may be aggregated and provided to the patient as a form and/or text excerpts 322 (also denoted as patient utterance summary, and/or patient utterance summary form). Utterances from other sections and/or subsections (e.g., OAP) not represented in the preliminary summary generated by the fine-tuned mSeq2SeqPLM model can be included. The patient utterance summary form may include, e.g., a checkbox next to each text excerpt and/or utterance 308-2. The checkbox in the patient utterance summary form may the enable the patient representative 302 (e.g., the patient) to opt out of allowing the medical professional 306 (e.g., the radiation oncologist) access to specific utterances 308-2.

The patient utterance summary form, the text excerpted 322, and/or the one or more checkboxes may be provided by a digital twin of the patient (also denoted as patient digital twin app), e.g., on a patient representative's 306 electronic device.

According to an embodiment, the patient representative 306 (e.g., the patient) is able to view health-related metrics and/or related documents via the patient digital twin app. The patient digital twin app can serve as a comprehensive patient health record. The digital twin app may display data on the patient's daily activities (e.g., exercise, and/or sleep), past medical and/or surgical history, social history, vital signs, lab test results, and/or medical imaging. Alternatively, or in addition, through the digital twin app, the patient representative 306 (e.g., the patient) may provide consent to which of his and/or her utterances 308-2 should be included with the verbatim report 318 (and/or the preliminary summary 326) and shown to the medical professional 304 (e.g., the radiation oncologist) after the medical examination 302 (also denoted as visit). The opt-out decision may only be possible for a predetermined period of time after the medical examination 302, and/or visit (e.g., for two days). If the predetermined period of time elapses without the patient representative 306 (e.g., the patient) selecting the checkboxes to accept, and/or omit, one or more utterances 308-2, according to an embodiment all utterances will be include in the preliminary medical examination report (also: preliminary summary).

In another embodiment, utterances in the verbatim report 318, and/or in a preliminary medical examination report (also: preliminary summary) 322; 326, may refer to, e.g., specific, medical imaging (also denoted as medical images in the context of the technique, and/or, e.g., including one or more CT scans, MRI scans, and/or Ultrasound scans) taken on one or more dates, in particular preceding the medical examination 302. The utterances 308-1; 308-2 in the verbatim report 318 may be analyzed by the entity linking model. The extracted results (e.g., imaging modality, site and/or date) may be used as an input query to a radiation oncology picture archiving and communication system (PACS), leading to retrieval of the medical images. The medical images may then be attached to the preliminary medical examination report 322; 326 (also: preliminary summary).

In another embodiment, the verbatim report 318, and/or the (e.g., preliminary) summary 322; 324 (e.g., without and/or with medical images) with the consented patient utterances are made available (e.g., in the method act S114) to the medical professional 304 (e.g., radiation oncologist) for verification. The medical professional 304 (e.g., radiation oncologist) can then review, and/or edit, the (e.g., preliminary) summary (and/or have another review of the medical images if needed) until he and/or she determines that the e.g., preliminary) summary is satisfactory. Then the medical professional 304 (e.g., radiation oncologist) may (e.g., as the method act S116) click a checkbox stating verification completed. He and/or she may then copy and paste (e.g., as the method act S118) the verified summary and/or medical examination report directly into the oncology information system (OIS) and/or EMR, e.g., as the final follow-up visit note.

The technique may include end-to-end learning. According to an embodiment, an interactive AI system (e.g., involving feedback from the medical professional 306, in particular radiation oncologist, in the acts S114 and S116, and optionally to some extent also from the patient representative 306, in the acts S110 and S112) may include training the, e.g., fine-tuned, mSeq2SeqPLM using reinforcement learning (RL) using a loss function, e.g., with rewards, computed out of the feedback.

The impact of other post-processing acts, such as entity recognition and/or linking component, may also be included in the loss function, e.g., reward computation. The corrections made by the medical professional 304 (e.g., the radiation oncologist) on the extracted entities in the summaries (and/or the, in particular preliminary, medical examination report 326) may force mSeq2SeqPLM to generate medical examination reports (also: summaries) with richer context, e.g., compared to conventional speech-recognition based techniques.

The most important takeaways from a medical examination 302 (also: physician-patient conversation) may often depend on the medical professional 304 (e.g., physician's) sub-specialty. In the exemplary case of radiation oncology, the reinforcement learning based systems 300 (and/or computing device 200) may focus on learning, e.g., on a local scale for a designated practice group, medical facility (also: hospital), and/or sub-specialty to provide more relevant medical examination reports (also: summaries), e.g., compared to conventional speech-recognition based techniques. In an embodiment, the RL agent (e.g., mSeq2SeqPLM) may be further customized using the attributes (e.g., designated practice group, medical facility, and/or sub-specialty) as, e.g., additional and/or supplementary, features. The RL agent may be fine-tuned, e.g., only, for close-domain (e.g., the sub-specialty and neighboring the sub-specialty) utterances-SOAP section and/or subsection pairs.

Alternatively, or in addition, the technique may include privacy by design. In an embodiment, once the verification completed box is checked (e.g., in the act of receiving S116 the user input by the medical professional 304), the sensor data 316—which may also be denoted as original verbal (and/or non-verbal) recordings-used to generate the medical examination report (also: summary), which may have been stored, e.g., in the medical facility's (also: healthcare institution's) private cloud governed by enterprise-level data privacy agreements, may be permanently deleted, as indicated at reference sign 328 in FIG. 3.

If the verification completed box is not checked within a predetermined period of time (which may be independently determined from the predetermined period of time for the user input by the patient representative 306) after the medical examination 302, and/or visit, (e.g., two days), according to an embodiment the preliminary medical examination report (also: preliminary summary) may be permanently deleted 328, e.g., in addition to the sensor data 316 (also: original recordings). According to another embodiment, which is combinable with the other embodiments, only the patient utterance summary 322 (also denoted as the patient representative's text excerpts) accessible via the patient digital twin app may persist. The patient utterance summary 322 may be retained and/or deleted at the discretion of the patient. While these strict data deletion measures would contribute towards preventing unauthorized access to protected health information (PHI) in the cloud, oncology information system (OIS), HER, and/or EMR, the patient representative 306 (e.g., the patient) may be simultaneously empowered to be responsible for protection of his, and/or her, utterances (also: narratives) 308-2 provided during the medical examination 302 (also: follow-up visit).

The technique can be distinguished from alternative methods for automated documentation in radiation oncology based on one or more of the following features. The technique may be multi-modal (e.g., including audio and/or text, video and/or images, including medical imaging, in particular medical imaging discussed during the medical examination 302), and/or multi-participant based visit notes can be generated by pre-training a (e.g., seq2seq) LM. Alternatively, or in addition, the technique may include the summarize-normalize paradigm utilizing the pretraining and fine-tuning of a multi-modal (e.g., seq2seq) LM (e.g., with an additional training objective of query masking). Further alternatively, or in addition, the technique may make use of medical entity linking (e.g., including mapping sequences to standardized medical concepts). A score may be used as reward, and/or loss function, for a reinforcement learning based end-to-end training of a pre-trained (e.g., seq2seq) LM. Further alternatively, or in addition, the technique may integrate a patient representative's (and/or patient's) consent for access to specific patient narratives via the opt-out checkboxes on the patient utterance summary form. Further alternatively, or in addition, the technique may include deleting 328 the original verbal and/or non-verbal recordings once verification completed has been checked by the radiation oncologist, and/or when a certain time period after the visit has elapsed.

Further alternatively, or in addition, the technique may make use of RL-based system that can help personalize the automated documentation of visit notes.

Wherever not already described explicitly, individual embodiments, or their individual aspects and features, described in relation to the drawings can be combined or exchanged with one another without limiting or widening the scope of the described invention, whenever such a combination or exchange is meaningful and in the sense of this invention. Advantages which are described with respect to a particular embodiment of present invention or with respect to a particular figure are, wherever applicable, also advantages of other embodiments of the present invention.

1. A computer-implemented method for generating a medical examination report from sensor data of a medical examination, the method comprising:
receiving sensor data from a set of sensors in relation to the medical examination, wherein the set of sensors comprises at least one microphone and the sensor data comprise at least audio data, wherein the audio data comprise utterances by a medical professional and a patient representative participating in the medical examination;
processing the received sensor data according to a type of sensors, wherein the processing comprises at least transforming the audio data into text;
generating a verbatim report of the medical examination based on the processed sensor data, wherein the verbatim report comprises each excerpt of the text being assigned to the medical professional or the patient representative;
converting the generated verbatim report into the medical examination report, wherein the medical examination report comprises a summary of the verbatim report, wherein the summary comprises vocabulary and/or text excerpts by accessing a predetermined ontology database in relation to a medical field of the medical examination; and
storing the medical examination report in an electronic medical report database.

2. The computer-implemented method according to claim 1, wherein the set of sensors comprises at least one further sensor selected from the group of:
a camera; and
a motion sensor.

3. The computer-implemented method according to claim 1, further comprising:
providing the text excerpts of the converted medical examination report, which are assigned to the patient representative, to the patient representative for approval; and
receiving a user input by the patient representative, wherein the user input is indicative of approval and/or rejection of the provided text excerpts assigned to the patient representative.

4. The computer-implemented method according to claim 1, further comprising:
providing the text excerpts of the converted medical examination report, which are assigned to the medical professional, to the medical professional for verification; and
receiving a user input, by the medical professional, indicative of a validation of the provided text excerpts assigned to the medical professional.

5. The computer-implemented method according to claim 1, further comprising:
temporarily storing intermediate data in a private cloud, wherein the intermediate data comprise the received sensor data, the generated verbatim report, and/or at least parts of the converted medical examination report; and
deleting the temporarily stored intermediate data from the private cloud after a predetermined period of time, after a rejection by the patient representative, and/or after a verification by the medical professional.

6. The computer-implemented method according to claim 1, wherein storing the medical examination report in an electronic medical report database comprises storing the medical examination report in a centralized memory of a medical facility.

7. The computer-implemented method according to claim 1, further comprising outputting the stored medical examination report.

8. The computer-implemented method according to claim 3, wherein storing the medical examination report in an electronic medical report database comprises storing the medical examination report in a digital twin of the patient, and/or wherein the providing of the text excerpts assigned to the patient representative and the receiving of the user input by the patient representative are performed using the digital twin of the patient.

9. The computer-implemented method according to claim 1, wherein the assignment of each excerpt of the text to the medical professional or the patient representative in generating the verbatim report is based on at least one of:
filtering of the audio data according to a frequency pattern, and/or according to a low pass filter, and/or a high pass filter, wherein the filtered audio data are classified according to characteristics determined by the filtering, and wherein the classifying characteristics are assigned to the medical professional and/or the patient representative;
extracting time stamps from the audio data and further sensor data, combining the audio data and the further sensor data according to the extracted time stamps, and assigning each excerpt of the text to the medical professional or the patient representative according to the combination of the audio data and the further sensor data per time stamp.

10. The computer-implemented method according to claim 1, further comprising:
identifying, based on the processed sensor data, medical imaging data that were examined during the medical examination; and
appending the medical imaging data to the medical examination report.

11. The computer-implemented method according to claim 1, wherein converting the generated verbatim report into a medical examination report is based on a trained artificial intelligence, AI, a trained neural network, NN, deep learning, DL, and/or reinforcement learning, RL.

12. The computer-implemented method according to claim 11, wherein the training of the AI and/or the NN, the DL, and/or the RL was based on training data sets, wherein each training data set comprised sensor data and an associated manually compiled medical examination report.

13. The computer-implemented method according to claim 12, wherein the training, and/or learning, was based on optimizing a loss function.

14. The computer-implemented method according to claim 1, wherein the medical examination comprises a radiology and/or an oncology examination.

15. A system for generating a medical examination report from sensor data of a medical examination, the system comprising:
a sensor interface configured to receive sensor data from a set of sensors in relation to a medical examination, wherein the sensor data comprise at least audio data, wherein the audio data comprise utterances by a medical professional and a patient representative participating in the medical examination;
a processor configured configured to:
process the received sensor data according to a type of sensor, wherein the processing comprises at least transforming the audio data into text;
generate a verbatim report of the medical examination based on the processed sensor data, wherein the verbatim report comprises each excerpt of the text being assigned to the medical professional or the patient representative; and
convert the generated verbatim report into the medical examination report, wherein the medical examination report comprises a summary of the verbatim report, wherein the summary comprises vocabulary, and/or text excerpts, by accessing a predetermined ontology database in relation to a medical field of the medical examination; and
a storage interface configured to forward the medical examination report to an electronic medical report database for storing.

16. The system of claim 15, further comprising:
the set of sensors comprising at least one microphone, wherein the set of sensors is configured to capture the sensor data, and wherein the audio data is captured by the at least one microphone.

17. The system of claim 15, further comprising:
the ontology database in relation to the medical field of the medical examination, wherein the ontology database comprises the vocabulary, and/or the text excerpts for the summary of the verbatim report, and wherein the ontology database is configured to be accessed for converting the generated verbatim report into the medical examination report.

18. The system of claim 15 further comprising a storage module configured to store the medical examination report in an electronic medical report database.

19. A non-transitory computer-readable medium on which program elements are stored that can be read and executed by a computer for generating a medical examination report from sensor data of a medical examination, the program elements comprising instructions to:
receive sensor data from a set of sensors in relation to the medical examination, wherein the set of sensors comprises at least one microphone and the sensor data comprise at least audio data, wherein the audio data comprise utterances by a medical professional and a patient representative participating in the medical examination;
process the received sensor data according to a type of sensor, wherein the processing comprises at least transforming the audio data into text;
generate a verbatim report of the medical examination based on the processed sensor data, wherein the verbatim report comprises each excerpt of the text being assigned to the medical professional or the patient representative;
convert the generated verbatim report into the medical examination report, wherein the medical examination report comprises a summary of the verbatim report, wherein the summary comprises vocabulary and/or text excerpts by accessing a predetermined ontology database in relation to a medical field of the medical examination; and
store the medical examination report in an electronic medical report database.
